(19) United States
(12) Patent Application Publication
(10) Pub. No.: US 2012/0039970 A1
(43) Pub. Date: Feb. 16, 2012

(54) READY-TO-USE, STABLE SUSPENSION OF PARTIALLY AMORPHOUS CAROTENOID PARTICLES

(75) Inventors: Christian Köpsel, Weinheim (DE); Clemens Sambale, Bohl-Iggelheim (DE); Andreas Hasse, Weisenheim/Sand (DE)

(73) Assignee: BASF SE, Ludwigshafen (DE)

(21) Appl. No.: 13/260,741

(22) PCT Filed: Mar. 26, 2010
(86) PCT No.: PCT/EP2010/053965

§ 371 Date: Oct. 21, 2011

(30) Foreign Application Priority Data
Mar. 30, 2009 (EP) 091566513

(51) Int. Cl. A61K 31/015; A61P 3/02; A61K 8/02; A61K 8/31; A61K 9/14

(52) U.S. Cl. 424/401; 514/763; 424/400

(57) ABSTRACT

The present invention relates to a ready-to-use stable suspension of partially amorphous carotenoid particles, a process for production thereof and also use thereof as additive to animal feeds, foods and food supplements and also cosmetic and pharmaceutical compositions.

The present invention relates to a ready-to-use stable suspension of partially amorphous carotenoid particles, a process for production thereof and also use thereof as additive to animal feeds, foods and food supplements and also cosmetic and pharmaceutical compositions.

The carotenoid class of substances is classified into two main groups, carotenes and xanthophylls. In contrast to the carotenes which are pure polyene hydrocarbons such as, for example, β-carotene or lycopene, oxygen functionalities such as hydroxyl, epoxy and/or carbonyl groups also occur in the xanthophylls. Typical members of this group are, inter alia astaxanthin, canthaxanthin, lutein and zeaxanthin.

These polyenes which can not only be obtained by synthesis but can also be isolated from natural sources are important colorants and active ingredients for the food and feed industries and also for the pharmaceutical sector and are of interest, e.g. because of their provitamin A activity.

Both carotenes and xanthophylls are insoluble in water whereas in fats and oils a likewise only low solubility is found. This restricted solubility and also the high sensitivity to oxidation are an obstacle to direct application of the relatively coarse-grained products obtained by chemical synthesis in the coloring of foods and feeds, since the substances in coarsely crystalline form are not storage-stable and give only poor coloring results. These effects which are disadvantageous for the practical use of carotenoids are especially expressed in an aqueous medium since they are virtually insoluble therein.

Improved color yields may only be achieved in the coloring of foods by specifically produced formulations in which the carotenoids are present in finely divided form and are optionally protected from oxidation by protective colloids. In addition, these formulations used in feeds lead to a higher bioavailability of the carotenoids that is the carotenes or xanthophylls, and thus indirectly to better coloring effects, e.g. in the pigmenting of egg yolks or fish.

To improve the color yields and to increase the absorbability and/or bioavailability, various processes have been described which all have the purpose of reducing the crystallite size of the active ingredients and bringing them to a particle size range of less than 10 μm.

Numerous methods described, inter alia, in Chimia 21, 329 (1967), WO 91/06292 and also in WO 94/19411, make use here of the grinding of carotenoids using a colloid mill and achieve thereby in part particle sizes of less than 2 μm.

WO 2007/003543 describes, for example, a grinding process in which β-carotene is comminuted to a particle size of about 0.6 μm as a suspension by grinding in the presence of sucrose or glucose and modified starch and subsequently converting the carotenoid-comprising suspension into a dry powder.

In addition to the grinding processes a number of combined emulsification/spray-drying processes exist such as those described, e.g. in DE-A-12 11 911 or in EP-A-0 410 236.

According to European patent EP-B-0 065 193, finely divided pulverulent carotenoid preparations are produced by dissolving a carotenoid in a volatile water-miscible organic solvent at elevated temperatures, if necessary at elevated pressure, precipitating the carotenoid by mixing the solution with an aqueous solution of a protective colloid and subsequently spray drying the solution.

A similar process for producing finely divided pulverulent carotenoid preparations is described in EP-A-0 937 412 with the use of water-immiscible solvents.

In the drinks industry, additives are generally added to the drinks in the form of liquid concentrates. Not only liquid formulations of colorants such as carotenoids are offered but also corresponding water-soluble pulverulent formulations from which, in the production process at the drinks manufacturers' premises, first an aqueous dispersion of the colorant is produced.

EP 0 239 086 describes emulsions of a carotenoid dissolved in oil, wherein, for stabilizing the oil droplets, a mixture of an ester of a long-chain fatty acid with ascorbic acid and a cold-water-soluble starch product such as, for example, starch octenyl succinate is used. The carotenoid concentration in these emulsions is between 0.1 and 2%.

In EP 0 551 638, stable liquid emulsions of fat-soluble vitamins or carotenoids are produced, wherein the outer phase is glycerol or a glycerol-water mixture and, as emulsifier and stabilizer, use is made of an ester of ascorbic acid with long-chain fatty acids. In the case of β-carotene, the preparations are distinguished by a brilliant yellow hue.

WO 93/04598 describes the production of a carotenoid composition which comprises a carotenoid in an oil, a dispersion of a water-dispersible matrix former, for example, a sugar and a stabilizer, for example gelatin or casein, and an emulsifier and also, optionally, a non-oily solvent such as glycerol.

WO 97/26802 describes ready-to-use water-dispersible pigment compositions in which a hydrophobic pigment of natural origin is present in dispersed form without addition of surfactants in an aqueous phase which comprises a hydrocolloid.

EP 0 795 585 describes aqueous, finely divided carotenoid suspensions which do not comprise a protective colloid, but comprise at least one special emulsifier. The color hue of the resultant suspensions varies owing to the differing particle size.

EP 0 832 569 describes the production of carotenoid-comprising, cold water-dispersible dry powders, wherein a dispersion of a finely divided carotenoid is thermally treated in order to set a desired color hue.

WO 2008/087140 describes liquid formulations comprising at least one carotenoid, at least one hydrophilic protective colloid and at least one water-miscible alcohol.

Commercially available pulverulent formulations of carotenoids have a good storage stability and demonstrate good coloring properties in the various drink uses, such as, for example, fruit juices, lemonades, sports drinks, milk drinks or vitamin drinks. For use in mineral-rich sports drinks or when using drinking water having a high content of calcium ions or magnesium ions, the bottled drinks must not show any ring formation (creaming) and also no deposition of the carotenoid component on the bottle wall.

Disadvantages in the use of pulverulent formulations of carotenoids are the risk of dust formation and, in conjunction therewith, contamination of the environment, the risk of electrostatic charging of the powders during transfer processes and also the risk of foaming or lump formation on dissolution of the powders in water. It is likewise disadvantageous that the pulverulent formulations of carotenoids are customarily used as aqueous stock solutions having a carotenoid content of about 0.1% by weight, as a result of which marked amounts of water are also always supplied to the food to be colored.

The object of the present invention was to provide a ready-to-use, stable formulation of carotenoids, which formulation has the desirable properties of the commercially available pulverulent formulations such as, for example, good storage stability with respect to microbial infestation, color hue constancy during storage, good thermal stability and insensitivity to the use in calcium- or magnesium-comprising drinks or when using drinking water with a high content of calcium ions or magnesium ions, but does not exhibit the processing disadvantages of a pulverulent formulation. A further object of the present invention was to provide a formulation of carotenoids having the above-mentioned properties, wherein the formulation can be used to produce an orange-colored and provitamin-A-enriched food, in particular a drink, and wherein the color hue should extend from yellowish orange through orange to reddish-orange.

This object is achieved by a ready-to-use, stable suspension of partially amorphous carotenoid particles, comprising:

| | |
|---|---|
| 1 to 20% by weight | of at least one carotenoid, |
| 0.2 to 20% by weight | of an edible oil, |
| 3 to 60% by weight | of a hydrophilic protective colloid, |
| 5 to 60% by weight | of a physiologically tolerated polyhydric |
| | alcohol and |
| 5 to 60% by weight | of water, |

wherein the percentages by weight relate to the ready-to-use, stable suspension, the sum of the fractions of the carotenoid, the edible oil, the protective colloid, the physiologically tolerated polyhydric alcohol and the water together is at least 80% by weight, and the weight ratio between carotenoid and edible oil ranges from 1:0.01 to 1:2.

The feature “ready-to-use”, in the context of the present invention, is taken to mean the property that the suspension according to the invention can be used directly for its purposes by the user, it can therefore be metered in directly, for example, in the food industry for coloring foods or for enriching foods with provitamin A (β-carotene), wherein, in the case of β-carotene, a color hue of yellowish orange to'reddish orange may be produced.

The feature “stable” in the context of the present invention, is taken to mean microbial, colloidal and chemical stability. Microbial stability is a bacteriostatic effect. The colloidal stability relates to the lack of occurrence of phase separation and changing of the color characteristics. Chemical stability relates to stable color characteristics and the carotenoid content.

The ready-to-use, stable suspension according to the invention comprises 1 to 20% by weight, preferably 3 to 15% by weight, particularly preferably 5 to 10% by weight, of a carotenoid.

Carotenoids which can be used in the ready-to-use, stable suspension according to the invention are the known representatives which can be obtained from natural sources or by synthesis. Examples thereof are β-carotene, lycopene, lutein, astaxanthin, zeaxanthin, cryptoxanthin, citranaxanthin, canthaxanthin, echinenone, bixin, β-apo-4-carotenal, β-apo-8-carotenal, esters of β-apo-4-carotenic acid, individually or as a mixture. Preferably, synthetically produced and purified carotenoids are used. Particularly preferably the carotenoid present in the ready-to-use, stable suspension according to the invention is selected from the group consisting of β-carotene, astaxanthin, canthaxanthin, citranaxanthin, lycopene and lutein. Very particularly preferably the carotenoid present in the ready-to-use, stable suspension according to the invention is β-carotene, which was produced in particular by synthesis.

The ready-to-use, stable suspension according to the invention comprises 0.2 to 20% by weight, preferably 0.5 to 10% by weight, particularly preferably 1 to 5% by weight, of an edible oil.

Edible oils which come into consideration are in principle physiologically tolerated oils of synthetic, mineral, vegetable or animal origin. Examples are sesame oil, corn germ oil, cottonseed oil, soybean oil, peanut oil, esters of medium-chain vegetable fatty acids, oleostearin, paraffin oil, glyceryl stearate, isopropyl myristate, diisopropyl adipate, cetyl stearyl 2-ethylhexanoate, hydrogenated polyisobutene, triglycerides of caprylic acid/capric acid, palm oil, palm kernel oil, lanolin and PUFAs (polyunsaturated fatty acids, such as eicosapentaenoic acid (EPA), docosahexaenoic acid (DHA) and alpha-linolenic acid.

Preferably, the edible oil in the ready-to-use, stable suspension according to the invention is of vegetable or animal origin, wherein it is liquid at 30° C., such as sunflower oil, palm oil, palm kernel oil, sesame oil, corn germ oil, cottonseed oil, soybean oil, peanut oil, esters of medium-chain triglycerides (called MCT oils), fish oils, such as mackerel, sprat or salmon oil.

Particularly preferably the edible oil in the ready-to-use, stable suspension according to the invention is a vegetable oil. Very particularly preferably, the edible oil is a medium-chain triglyceride (MCT oil). Medium-chain triglycerides comprise very largely only saturated fatty acids such as are present, for example, in palm kernel oil or coconut oil.

In the ready-to-use, stable suspension according to the invention, the weight ratio between carotenoid and edible oil ranges from 1:0.01 to 1:2, preferably from 1:0.05 to 1:1.5, particularly preferably from 1.0.2 to 1:1, in particular from 1:0.3 to 1:0.7. In the case of the described weight ratios of carotenoid to edible oil, customarily more than 50% of the carotenoid used is present as solid and a small fraction, that is to say less than 50%, of the carotenoid used is dissolved in the edible oil which is present.

The carotenoid and the edible oil form the main component of the hydrophobic phase of the suspension according to the invention, wherein according to the current state of knowledge, one part of the oil is associated with the solid carotenoid particles and one part of the oil is present in droplet form, wherein these oil droplets comprise only molecularly dissolved carotenoid and no carotenoid particles.

The solid carotenoid particles are not completely crystalline, but they are partially amorphous. In the present invention, partially amorphous is taken to mean a degree of crystallinity of less than 95%, preferably a degree of crystallinity of less than 70%. The crystalline and amorphous fraction in the carotenoid particles can be determined, for example by X-ray diffraction measurements. The X-ray amorphous fraction in the carotenoid particles of the suspension according to the invention is preferably between 50 and 100%, in particular between 60 and 100%.

The ready-to-use, stable suspension according to the invention comprises 3 to 60% by weight, preferably 5 to 40% by weight, in particular 7 to 30% by weight of a hydrophilic protective colloid.

As hydrophilic protective colloid, colloids which are water-soluble or swellable in water come into consideration. Preferably, the protective colloids are protein-based protective colloids, modified starch derivatives and modified cellulose derivatives. Protein-based protective colloids are, in particular casein, caseinate, bovine gelatin, pig gelatin or fish gelatin, in particular gelatin broken down under acidic or basic conditions having Bloom values in the range from 0 to 250 and also mixtures thereof. The modified starch derivatives and cellulose derivatives are in particular, the esters, for example, starch octenyl succinate. Corresponding products are commercially available under the name Purity Gum 2000 from National Starch or Clear Gum CO 01 from Roquette, Hi Cap 100 or Capsul from National Starch.

Preferably, the ready-to-use, stable suspension according to the invention, as hydrophilic protective colloid, comprises a chemically modified starch.

Chemically modified starch is taken to mean chemically and/or enzymatically produced conversion products of starch. These can be starch ethers, starch esters or starch phosphates. Preferred representatives of this group are starch esters, in particular octenyl succinate starch such as, for example, Capsul® (sodium octenyl succinate starch) from National Starch, Cleargum CO 01 from Roquette or Purity® Gum 2000 (sodium octenyl succinate starch) from National Starch, in particular a sodium octenyl succinate starch such as Purity® Gum 2000.

Particularly preferably, the ready-to-use stable suspension according to the invention comprises octenyl succinate starch as hydrophilic protective colloid.

The ready-to-use, stable suspension according to the invention comprises 5 to 60% by weight, preferably 15 to 50% by weight, particularly preferably 25 to 45% by weight, in particular 30 to 40% by weight of a physiologically tolerated polyhydric alcohol.

The physiologically tolerated polyhydric alcohol present in the suspension according to the invention customarily has a boiling point of above 150° C., preferably above 180° C., in particular above 200° C. at atmospheric pressure.

The physiologically tolerated polyhydric alcohol is preferably glycerol, monoesters of glycerol with C₁-C₅-monocarboxylic acids, monoethers of glycerol, propylene glycol or sorbitol. Particularly preferably, glycerol is the physiologically tolerated polyhydric alcohol.

The ready-to-use, stable suspension according to the invention comprises 5 to 60% by weight, preferably 15 to 50% by weight, particularly preferably 25 to 45% by weight, in particular 30 to 40% by weight of water.

In the ready-to-use, stable suspension according to the invention, the sum of the fractions of the carotenoid, the edible oil, the protective colloid, the physiologically tolerated polyhydric alcohol and the water together is at least 80% by weight, preferably at least 90% by weight, in particular at least 95% by weight.

In the ready-to-use, stable suspension according to the invention, the content of a further substance having emulsifying activity from the class of surfactants (low-molecular-weight compounds) in addition to the hydrophilic protective colloid (high-molecular-weight compound or polymer) is customarily less than 2% by weight, preferably less than 1% by weight. Surfactants as substances having emulsifying activity are, for example, ascorbyl palmitate, polyglycerol esters of fatty acids such as polyglycerol 3-polyricinoleate (PGPR 90), sorbitan esters of fatty acids such as sorbitan monostearate (Span 60), PEG(20)-sorbitol monooleate, propylene glycol esters of fatty acids, sucrose esters of long-chain fatty acids, such as for example, sucrose monopalmitate, or phospholipids, such as lecithin.

If the further substance having emulsifying activity is ascorbyl palmitate, the content of ascorbyl palmitate is preferably less than 0.5% by weight, particularly preferably less than 0.25% by weight, in particular less than 0.1% by weight, wherein the percentages by weight relate to the total weight of the suspension.

For increasing the stability of the carotenoid in the ready-to-use, stable suspension according to the invention to oxidative decay, it is advantageous to add stabilizers such as d,l-alpha-tocopherol, tertiary butylated hydroxytoluene, tertiary butylated hydroxyanisole, ascorbic acid, salts of ascorbic acid or ethoxyquin as antioxidant.

Preferably, the ready-to-use, stable suspension of partially amorphous carotenoid particles according to the invention comprises additionally 0.1 to 5% by weight, preferably 0.2 to 4% by weight, of at least one antioxidant, wherein the percentages by weight relate to the ready-to-use, stable suspension. Particularly preferably, 0.5 to 2.0% by weight of d,l-alpha-tocopherol is added as antioxidant to the ready-to-use, stable suspension according to the invention.

Particular preference is given to a ready-to-use, stable suspension according to the invention comprising,

| | |
|---|---|
| 3 to 10% by weight | of β-carotene as carotenoid, |
| 1 to 5% by weight | of medium-chain triglyceride as edible oil, |
| 0.5 to 2.0% by weight | of d,l-alpha-tocopherol |
| 10 to 24% by weight | of octenyl succinate starch as hydrophilic |
| | protective colloid, |
| 30 to 40% by weight | of glycerol as physiologically tolerated |
| | polyhydric alcohol and |
| 30 to 40% by weight | of water, |

wherein the sum of the fractions of β-carotene, medium-chain triglyceride, d,l-alpha-tocopherol, octenyl succinate starch, glycerol and water together is at least 95% by weight, and the weight ratio between β-carotene and medium-chain triglyceride ranges from 1:0.3 to 1:0.7.

In the ready-to-use, stable suspension of partially amorphous carotenoid particles according to the invention the partially amorphous carotenoid particles are preferably present as nanoparticulate particles.

Nanoparticulate particles are taken to mean those particles which have a median particle size D[4,3] determined by Fraunhofer diffraction from 0.02 to 100 μm, preferably 0.05 to 50 μm, particularly preferably 0.05 to 20 μm, very particularly preferably 0.05 to 5 μm, in particular 0.05 to 1.0 μm. The expression D[4,3] denotes the volume-weighted median diameter (see handbook for the Malvern Mastersizer S, Malvern Instruments Ltd., UK) which can be determined by means of Fraunhofer diffraction.

Preferably, the carotenoid particles in the suspension according to the invention have a median particle size D[4,3] of 0.05 to 0.8 μm, preferably 0.1 to 0.7 μm, in particular 0.4 to 0.6 μm.

Particularly preferably, the carotenoid particles in the ready-to-use, stable suspension according to the invention have a median particle size of 0.05 to 0.8 μm, preferably 0.1 to 0.7 μm, in particular 0.4 to 0.6 μm and have a mean X-ray amorphous fraction between 50 and 100%, preferably between 60 and 100%.

The invention also further relates to a process for producing a ready-to-use stable suspension of partially amorphous carotenoid particles comprising:

| | |
|---|---|
| 1 to 20% by weight | of at least one carotenoid, |
| 0.2 to 20% by weight | of an edible oil, |
| 3 to 60% by weight | of a hydrophilic protective colloid, |
| 5 to 60% by weight | of a physiologically tolerated polyhydric |
| | alcohol and |
| 5 to 60% by weight | of water, |

wherein the percentages by weight relate to the ready-to-use, stable suspension, the sum of the fractions of the carotenoid, the edible oil, the protective colloid, the physiologically tolerated polyhydric alcohol and the water together is at least 80% by weight, and the weight ratio between carotenoid and edible oil ranges from 1:0.01 to 1:2
comprising the steps
a₁) dissolving the carotenoids together with the edible oil in a water-miscible organic solvent or in a mixture of water and a water-miscible organic solvent at temperatures above 30° C., or
a₂) dissolving the carotenoids together with the edible oil in a water-immiscible organic solvent,
b) mixing the solution obtained according to a₁) or a₂) with a molecularly dispersed or colloidally dispersed solution of the protective colloid in a mixture of water and the physiologically tolerated polyhydric alcohol, wherein the carotenoid-comprising hydrophobic phase is produced as nanodisperse phase,
c) removing the organic solvent used in a₁) or a₂) and concentrating the suspension formed to the desired content of carotenoid.

Preferred embodiments with respect to the components carotenoid, edible oil, hydrophilic protective colloid, physiologically tolerated polyhydric alcohol and water and also the amounts used thereof may be found in the explanations already made at the outset.

The water-miscible solvents used in stage a₁) of the process according to the invention are primarily water-miscible, thermally stable, volatile solvents comprising only carbon, hydrogen and oxygen such as alcohols, ethers, esters, ketones or acetals. Expediently, use is made of those solvents which are at least up to 10% water-miscible, have a boiling point below 200° C. and/or have fewer than 10 carbon atoms. Particularly preferably, use is made of methanol, ethanol, n-propanol, isopropanol, 1,2-butanediol 1-methyl ether (1-methoxy-2-butanol), 1,2-propanediol 1-n propyl ether (1-propoxy-2-propanol), tetrahydrofuran or acetone.

The expression “a water-immiscible organic solvent” in the context of the present invention, is an organic solvent having a water solubility at atmospheric pressure of less than 10%. Possible solvents which come into consideration in this case are, inter alia, halogenated aliphatic hydrocarbons such as, e.g. methylene chloride, chloroform and carbon tetrachloride, esters of carboxylic acid such as dimethyl carbonate, diethyl carbonate, propylene carbonate, ethyl formate, methyl, ethyl or isopropyl acetate and also ethers such as methyl tert-butyl ether. Preferred water-immiscible organic solvents are the following compounds from the group consisting of dimethyl carbonate, propylene carbonate, ethyl formate, ethyl acetate, isopropyl acetate and methyl tert-butyl ether.

According to the invention, preferably process step a₁) is carried out, wherein the carotenoid is dissolved together with the edible oil in a water-miscible, organic solvent, or in a mixture of water and a water-miscible, organic solvent at temperatures above 30° C., preferably between 50° C. and 240° C., in particular 100° C. to 200° C., particularly preferably 140° C. to 180° C., if necessary under pressure.

Since the action of high temperatures in some circumstances can decrease the desired high all-trans-isomer fraction of the carotenoid, in particular also of β-carotene, the carotenoid(s) is/are dissolved as rapidly as possible, for example, in the region of seconds, e.g. in 0.1 to 10 seconds, particularly preferably in less than 1 second. For rapid production of the molecularly dispersed solution, the use of elevated pressure, e.g. in the range from 20 bar to 80 bar, preferably 30 to 60 bar, can be advantageous.

The resultant molecularly dispersed solution is subsequently admixed in process step b) directly with the possibly cooled molecularly dispersed or colloidally dispersed solution of the protective colloid in a mixture of water and the physiologically tolerated polyhydric alcohol, wherein the solvent component from process step a₁) is transferred to the aqueous phase and the hydrophobic phase of the carotenoids together with the edible oil is formed as nanodisperse phase. Preferably, in process step b), a mixture temperature of about 35° C. to 80° C. is established.

With respect to a more detailed description of process and apparatus for the abovementioned dispersion, reference is made at this point to EP-B-0 065 193.

In the process according to the invention, in process step c) the organic solvents used in process step a₁) or a₂) are removed and the suspension formed is concentrated to the desired content of carotenoid by removing excess water. The physiologically tolerated polyhydric alcohol used is virtually not removed in this concentration step due to its high boiling point.

The ready-to-use, stable suspension of partially amorphous carotenoid particles according to the invention is distinguished in that it is significantly more concentrated with respect to the carotenoid content compared with the aqueous stock solutions which can be produced from pulverulent carotenoid formulations, and thus can also be added to drink bases which only tolerate very small amounts of water. The suspension according to the invention may be metered in without problem and shows no unwanted sedimentation.

In addition, the suspension according to the invention has a good storage stability with respect to microbiological infestation and with respect to color hue constancy during storage. The suspension according to the invention, in addition, shows good thermal stability and the required insensitivity in use in calcium- or magnesium-comprising drinks or when using drinking water having a high content of calcium ions or magnesium ions. The drinks admixed with the suspension according to the invention have a good stability with respect to unwanted ring formation (creaming).

The ready-to-use, stable suspension of partially amorphous carotenoid particles according to the invention is suitable, inter alia, as additive to food preparations, for example for coloring foods such as drinks, as compositions for producing pharmaceutical and cosmetic preparations and also for producing food supplement formulations, for example, multivitamin formulations in the human and animal sectors. Preferably, the ready-to-use, stable suspension according to the invention is suitable as an additive to drinks.

The suspension according to the invention, in the case of the carotenoid β-carotene, has an orange color hue and is suitable for coloring foods in the color range yellowish-orange to reddish-orange. The β-carotene-comprising suspension in addition serves for enriching foods with provitamin A.

Therefore the invention also further relates to the use of the above-described ready-to-use stable suspension of partially amorphous carotenoid particles according to the invention as additive to animal feeds, foods, food supplements and also cosmetic and pharmaceutical compositions, in particular also as additive in the production of drinks, wherein the suspension according to the invention is preferably added directly to the drink.

The present invention likewise relates to animal feeds, foods and food supplements, in particular a drink which comprises the ready-to-use, stable suspension of partially amorphous carotenoid particles according to the invention.

The present invention further relates to the use of the above-described ready-to-use, stable suspension wherein the carotenoid is β-carotene for enriching animal feeds, foods and food supplements and also cosmetic and pharmaceutical compositions with provitamin A

The present invention likewise relates to a process for enriching animal feeds, foods and food supplements and also cosmetic and pharmaceutical compositions, in particular drinks, with provitamin A by adding to same the ready-to-use, stable suspension of partially amorphous carotenoid particles according to the invention wherein the carotenoid is β-carotene.

The invention will be explained by the examples hereinafter which do not restrict the invention, however:

### EXAMPLES

### Example 1

### Production of a β-Carotene Suspension

In a heatable receiving flask, at a temperature of 30° C., 57 g of crystalline β-carotene, 27 g of MCT oil and 8.4 g of α-tocopherol were suspended in 315 g of an azeotropic isopropanol/water mixture. The active ingredient suspension was then heated to 73° C. and, at a flow rate of 3.4 kg/h, continuously mixed with further isopropanol/water azeotrope of the temperature 235° C. and a flow rate of 4.8 kg/h in a mixing chamber, wherein the β-carotene dissolved at a mixture temperature which was established at 171° C. at a pressure of 65 bar. This active ingredient solution was immediately mixed in a second mixing chamber with an aqueous solution of 227 g of Purity Gum 2000, 286 g of glycerol in 6646 g of distilled water at a flow rate of 60 kg/h.

The active ingredient particles which resulted on mixing had, in the isopropanol/water mixture, a particle size of 438 nm at an E1/1 value of 115 (E1/1: extinction of a 1% strength by weight suspension in a 1 cm cuvette).

Subsequently, the active ingredient suspension was concentrated to a concentration of approximately 6.2% by weight of active ingredient content on a thin-film evaporator.

The suspension produced in example 1 which had a β-carotene content of approximately 6.2% by weight was denoted by the name OL in the use studies hereinafter.

### Example 2

### Sports Drink without Fruit Juice (15 ppm of β-Carotene)

| Ingredients | Amount used in g/l |
|---|---|
| Ascorbic acid | 0.150 |
| Calcium hydrogenphosphate | 0.215 |
| Carboxymethylcellulose | 2.000 |
| Potassium dihydrogenphosphate | 0.350 |
| Trisodium citrate | 0.486 |
| Sodium benzoate | 0.150 |
| Potassium sorbate | 0.200 |
| Citric acid (anhydrous) | 2.500 |
| Sucrose | 61.000 |
| H₂O | to make up to 1000 |
| OL calculated as β-carotene | 0.015 |

After blending the components the drink was pasteurized for 60 s at 90° C.

### Comparative Example 2b

A sports drink was prepared in a similar manner as described in example 2, wherein instead of the suspension OL of example 1 according to the invention, use was made of a stock solution having a β-carotene content of 0.1% by weight, prepared from the pulverulent commercially available product Lucarotin® 10 CWD/O (10% by weight of β-carotene), as β-carotene source.

### Result:

It was observed that the chemical stability (beta-carotene content, color) of the sports drink produced in example 2 had no disadvantage compared with the sports drink produced in comparative example 2b.

### Example 3

### Milk Drink (5 ppm of β-Carotene)

| Ingredients | Amount used in g/l |
|---|---|
| H₂O | to make up to 1000 |
| Orange juice concentrate (54° Brix) | 15.046 |
| Low-fat milk 0.1% fat | 3.636 |
| Stabilizer pectin | 3.314 |
| Citric acid (anhydrous) | 6.179 |
| Malic acid | 1.612 |
| Calcium chloride | 1.075 |
| Trisodium citrate | 1.881 |
| Sucrose | 107.464 |
| Potassium sorbate | 0.445 |
| Sodium benzoate | 0.493 |
| OL calculated as β-carotene | 0.005 |

After blending the drink was homogenized at high pressure at 200 bar and pasteurized for 60 s at 90° C.

### Example 4

### Alcopop (3.5 ppm of β-Carotene)

| Ingredients | Amount used in g/l |
|---|---|
| Neutral spirit (23.8% Vol.) | 188.810 |
| Sucrose | 49.260 |
| Orange juice concentrate (54° Brix) | 98.820 |
| Citric acid | 1.000 |
| OL calculated as β-carotene | 0.0035 |
| Ascorbic acid | 0.150 |
| Sodium benzoate | 0.500 |
| Potassium sorbate | 0.500 |
| H₂O | to make up to 1000 |

After blending the drink was homogenized at high pressure at 200 bar and pasteurized for 60 s at 65° C.

### Comparative Example 4b

An alcopop was prepared in a similar manner as described in example 4, wherein instead of the suspension OL from example 1 according to the invention use was made of a stock solution having a β-carotene content of 0.1% by weight, prepared from the pulverulent commercially available product Lucarotin® 10 CWD/O, as β-carotene source.

### Result:

It was observed that the chemical stability (beta-carotene content, color) of the alcopop produced in example 4 had no disadvantage compared with the alcopop produced in comparative example 4b.

### Example 5

### Vitamin Drink Having 30% Fruit Juice (15 ppm of β-Carotene)

| Ingredients | Amount used in g/l |
|---|---|
| Sucrose | 85.300 |
| Pectin | 0.200 |
| Sodium benzoate | 0.200 |
| Potassium sorbate | 0.200 |
| Ascorbic acid | 0.300 |
| Citric acid 50% by weight | 5.000 |
| Calcium lactate-5-hydrate | 4.500 |
| H₂O | to make up to 1000 |
| Orange juice concentrate (54° Brix) | 64.230 |
| OL calculated as β-carotene | 0.015 |
| Vitamin E 500 BG | 0.070 |

After blending the drink was homogenized at high pressure at 200 bar and pasteurized for 60 s at 90° C.

### Example 6

### Vitamin Drink Having 100% Fruit Juice (30 ppm of β-Carotene)

| Ingredients | Amount used in g/l |
|---|---|
| Orange juice concentrate (54° Brix) | 220.000 |
| H₂O | to make up to 1000 |
| Sodium benzoate | 1.000 |
| Ascorbic acid | 0.400 |
| OL calculated as β-carotene | 0.03 |

After blending the drink was homogenized at high pressure of 100 bar and pasteurized for 60 s at 90° C.

### Example 7

### Orange Juice Base in the Cold-Blend Process (30 ppm of β-Carotene)

| Ingredients | Amount used in g/l |
|---|---|
| Orange juice concentrate (54° Brix) | 840.000 |
| Sodium benzoate | 3.500 |
| H₂O | to make up to 1000 |
| Ascorbic acid | 1.500 |
| OL calculated as β-carotene | 0.030 |

In the cold blend process all ingredients for producing the orange juice base were incorporated into orange juice concentrate at −2° C. The high viscosity of the drinks medium and also the low temperature made special demands of the β-carotene formulation with respect to stability and processability.

### Example 8

### Salad Dressing (6.2 ppm of β-Carotene)

| Ingredients | Amount used in g/l |
|---|---|
| Xanthan | 30.000 |
| Mustard powder | 12.500 |
| Sodium chloride | 40.000 |
| Vinegar | 90.000 |
| Sucrose | 115.000 |
| H₂O | to make up to 1000 |
| Vegetable oil | 360.000 |
| OL calculated as β-carotene | 0.0062 |

The ingredients were mixed, homogenized with an Ultra-Turrax and heated.

### Example 9

### Pudding (2 ppm of β-Carotene)

| Ingredients | Amount used in g/l |
|---|---|
| Corn starch | 64.000 |
| Sucrose | 72.000 |
| Milk 3.5% fat | to make up to 1000 |
| OL calculated as β-carotene | 0.002 |

### Example 10

### Ice Preparation (3 ppm of β-Carotene)

| Ingredients | Amount used in g/l |
|---|---|
| Milk 3.5% fat | to make up to 1000 |
| Cream 34% fat | 43.103 |
| Sucrose | 196.55 |
| Emulsifier lecithin | 2.586 |
| Skimmed milk powder | 41.379 |
| Sodium chloride | 0.862 |
| OL calculated as β-carotene | 0.003 |

### Example 11

### Yoghurt (5 ppm of β-Carotene)

| Ingredients | Amount used in g/l |
|---|---|
| Milk 3.5% fat | to make up to 1000 |
| Yoghurt | 150.000 |
| OL calculated as β-carotene | 0.005 |

1. A ready-to-use, stable suspension of partially amorphous carotenoid particles comprising

| | |
|---|---|
| 1 to 20% by weight | of a carotenoid, |
| 0.2 to 20% by weight | of an edible oil, |
| 3 to 60% by weight | of a hydrophilic protective colloid, |
| 5 to 60% by weight | of a physiologically tolerated polyhydric |
| | alcohol and |
| 5 to 60% by weight | of water, |

wherein the percentages by weight expressed are relative to the ready-to-use, stable suspension; the sum of the fractions of the carotenoid, the edible oil, the hydrophilic protective colloid, the physiologically tolerated polyhydric alcohol and the water together is at least 80% by weight of the ready-to-use, stable suspension of partially amorphous carotenoid particles, and the weight ratio between carotenoid and edible oil is 1:0.01 to 1:2.

2. The ready-to-use, stable suspension of partially amorphous carotenoid particles of claim 1, wherein the carotenoid is selected from the group consisting of β-carotene, astaxanthin, canthaxanthin, citranaxanthin, lycopene and lutein.

3. The ready-to-use, stable suspension of partially amorphous carotenoid particles of claim 1, wherein the carotenoid is β-carotene.

4. The ready-to-use, stable suspension of partially amorphous carotenoid particles of claim 1, wherein the edible oil is a vegetable oil.

5. The ready-to-use, stable suspension of partially amorphous carotenoid particles of claim 1, wherein the physiologically tolerated polyhydric alcohol is glycerol.

6. The ready-to-use, stable suspension of partially amorphous carotenoid particles of claim 1, wherein the hydrophilic protective colloid is a chemically modified starch.

7. The ready-to-use, stable suspension of partially amorphous carotenoid particles of claim 6, wherein the chemically modified starch is octenyl succinate starch.

8. The ready-to-use, stable suspension of partially amorphous carotenoid particles of claim 1, further comprising 0.1 to 5% by weight of an antioxidant.

9. The ready-to-use, stable suspension of partially amorphous carotenoid particles of claim 1 comprising,
3 to 10% by weight of β-carotene as the carotenoid,
1 to 5% by weight of medium-chain triglyceride as the edible oil,
0.5 to 2.0% by weight of d,l-alpha-tocopherol
10 to 24% by weight of octenyl succinate starch as the hydrophilic protective colloid,
30 to 40% by weight of glycerol as the physiologically tolerated polyhydric alcohol and
30 to 40% by weight of the water,
wherein the sum of the fractions of β-carotene, medium-chain triglyceride, d,l-alpha-tocopherol, octenyl succinate starch, glycerol and water together is at least 95% by weight of the ready-to-use, stable suspension of partially amorphous carotenoid particles, and the weight ratio between β-carotene and medium-chain triglyceride is 1:0.3 to 1:0.7.

10. The ready-to-use, stable suspension of partially amorphous carotenoid particles of claim 1, wherein the carotenoid particles have a median particle size of 0.05 μm to 0.8 μm and have a mean X-ray amorphous fraction of 50% to 100%.

11. A process for producing a ready-to-use stable suspension of partially amorphous carotenoid particles comprising:
1 to 20% by weight of a carotenoid,
0.2 to 20% by weight of an edible oil,
3 to 60% by weight of a hydrophilic protective colloid,
5 to 60% by weight of a physiologically tolerated polyhydric alcohol and
5 to 60% by weight of water,
wherein the percentages by weight expressed are relative to the ready-to-use, stable suspension, the sum of the fractions of the carotenoid, the edible oil, the protective colloid, the physiologically tolerated polyhydric alcohol and the water together is at least 80% by weight of the ready-to-use, stable suspension of partially amorphous carotenoid particles, and the weight ratio between carotenoid and edible oil is 1:0.01 to 1:2, comprising the steps of
a₁) dissolving the carotenoid and the edible oil in a water-miscible organic solvent or in a mixture of water and a water-miscible organic solvent at a temperature above 30° C., or
a₂) dissolving the carotenoid and the edible oil in a water-immiscible organic solvent,
b) mixing the solution obtained in a₁) or a₂) with a molecularly dispersed or colloidally dispersed solution of the hydrophilic protective colloid in a mixture of water and the physiologically tolerated polyhydric alcohol, wherein the carotenoid-comprising hydrophobic phase is produced as a nanodispersed hydrophobic phase,
c) removing the organic solvent used in a₁) or a₂) and concentrating the suspension formed.

12. A pharmaceutical composition comprising the ready-to-use, stable suspension of partially amorphous carotenoid particles of claim 1.

13. A cosmetic composition comprising the ready-to-use, stable suspension of partially amorphous carotenoid particles of claim 1.

14. (canceled)

15. An animal feed, food or food supplement comprising the ready-to-use, stable suspension of partially amorphous carotenoid particles of claim 1.

16. A drink comprising the ready-to-use, stable suspension of partially amorphous carotenoid particles of claim 1.

17. (canceled)

18. A process for enriching a product with provitamin A comprising adding the ready-to-use, stable suspension of partially amorphous carotenoid particles of claim 3 to the product, wherein the product is selected from the group consisting of animal feeds, foods, food supplements, cosmetic compositions and pharmaceutical compositions.

19. The process of claim 18, wherein the product is a drink.
